SEQUENCE LISTING (57 sequences)

SEQ ID NO: 1 - DNA, 19 bp, Artificial Sequence
  Chemically synthesized forward primer for amplifying enteroviral RNA
cccctgaatgcggctaatc

SEQ ID NO: 2 - DNA, 20 bp, Artificial Sequence
  Chemically synthesized reverse primer for amplifying enteroviral RNA
aaggaaacacggacacccaa

SEQ ID NO: 3 - DNA, 25 bp, Artificial Sequence
  Chemically synthesized probe for enteroviral RNA
tccgccacagacttgcgcattacga

SEQ ID NO: 4 - DNA, 25 bp, Artificial Sequence
  Chemically synthesized probe for enteroviral RNA
tccgctgcagagttgcccgttacga

SEQ ID NO: 5 - DNA, 25 bp, Artificial Sequence
  Chemically synthesized probe for enteroviral RNA
tccgctgcrgagttrcccrttacga

SEQ ID NO: 6 - DNA, 17 bp, Artificial Sequence
  Chemically synthesized forward primer for amplifying enteroviral RNA
cccctgaatgcggctaa

SEQ ID NO: 7 - DNA, 17 bp, Artificial Sequence
  Chemically synthesized forward primer for amplifying enteroviral RNA
cctgaatgcggctaatc

SEQ ID NO: 8 - DNA, 15 bp, Artificial Sequence
  Chemically synthesized forward primer for amplifying enteroviral RNA
cctgaatgcggctaa

SEQ ID NO: 9 - DNA, 17 bp, Artificial Sequence
  Chemically synthesized forward primer for amplifying enteroviral RNA
ccctgaatgcggctaat

SEQ ID NO: 10 - DNA, 18 bp, Artificial Sequence
  Chemically synthesized forward primer for amplifying enteroviral RNA
ccctgaatgcggctaatc

SEQ ID NO: 11 - DNA, 18 bp, Artificial Sequence
  Chemically synthesized forward primer for amplifying enteroviral RNA
cccctgaatgcggctaat

SEQ ID NO: 12 - DNA, 16 bp, Artificial Sequence
  Chemically synthesized forward primer for amplifying enteroviral RNA
cctgaatgcggctaat

SEQ ID NO: 13 - DNA, 17 bp, Artificial Sequence
  Chemically synthesized forward primer for amplifying enteroviral RNA
ccctgaatgcggctaat

SEQ ID NO: 14 - DNA, 20 bp, Artificial Sequence
  Chemically synthesized forward primer for amplifying enteroviral RNA
cccctgaatgcggctaatct

SEQ ID NO: 15 - DNA, 20 bp, Artificial Sequence
  Chemically synthesized forward primer for amplifying enteroviral RNA
gcccctgaatgcggctaatc

SEQ ID NO: 16 - DNA, 25 bp, Artificial Sequence
  Chemically synthesized forward primer for amplifying enteroviral RNA
gaattccccctgaatgcggctaatc

SEQ ID NO: 17 - DNA, 18 bp, Artificial Sequence
  Chemically synthesized reverse primer for amplifying enteroviral RNA
aaggaaacacggacaccc

SEQ ID NO: 18 - DNA, 18 bp, Artificial Sequence
  Chemically synthesized reverse primer for amplifying enteroviral RNA
ggaaacacggacacccaa

SEQ ID NO: 19 - DNA, 16 bp, Artificial Sequence
  Chemically synthesized reverse primer for amplifying enteroviral RNA
ggaaacacggacaccc

SEQ ID NO: 20 - DNA, 18 bp, Artificial Sequence
  Chemically synthesized reverse primer for amplifying enteroviral RNA
aggaaacacggacaccca

SEQ ID NO: 21 - DNA, 19 bp, Artificial Sequence
  Chemically synthesized reverse primer for amplifying enteroviral RNA
aggaaacacggacacccaa

SEQ ID NO: 22 - DNA, 19 bp, Artificial Sequence
  Chemically synthesized reverse primer for amplifying enteroviral RNA
aaggaaacacggacaccca

SEQ ID NO: 23 - DNA, 17 bp, Artificial Sequence
  Chemically synthesized reverse primer for amplifying enteroviral RNA
ggaaacacggacaccca

SEQ ID NO: 24 - DNA, 17 bp, Artificial Sequence
  Chemically synthesized reverse primer for amplifying enteroviral RNA
aggaaacacggacaccc

SEQ ID NO: 25 - DNA, 21 bp, Artificial Sequence
  Chemically synthesized reverse primer for amplifying enteroviral RNA
aaggaaacacggacacccaaa

SEQ ID NO: 26 - DNA, 21 bp, Artificial Sequence
  Chemically synthesized reverse primer for amplifying enteroviral RNA
aaaggaaacacggacacccaa

SEQ ID NO: 27 - DNA, 26 bp, Artificial Sequence
  Chemically synthesized reverse primer for amplifying enteroviral RNA
gaattcaaggaaacacggacacccaa

SEQ ID NO: 28 - DNA, 23 bp, Artificial Sequence
  Chemically synthesized probe for enteroviral RNA
tccgccacagacttgcgcattac

SEQ ID NO: 29 - DNA, 23 bp, Artificial Sequence
  Chemically synthesized probe for enteroviral RNA
cgccacagacttgcgcattacga

SEQ ID NO: 30 - DNA, 21 bp, Artificial Sequence
  Chemically synthesized probe for enteroviral RNA
cgccacagacttgcgcattac

SEQ ID NO: 31 - DNA, 23 bp, Artificial Sequence
  Chemically synthesized probe for enteroviral RNA
ccgccacagacttgcgcattacg

SEQ ID NO: 32 - DNA, 24 bp, Artificial Sequence
  Chemically synthesized probe for enteroviral RNA
ccgccacagacttgcgcattacga

SEQ ID NO: 33 - DNA, 24 bp, Artificial Sequence
  Chemically synthesized probe for enteroviral RNA
tccgccacagacttgcgcattacg

SEQ ID NO: 34 - DNA, 22 bp, Artificial Sequence
  Chemically synthesized probe for enteroviral RNA
cgccacagacttgcgcattacg

SEQ ID NO: 35 - DNA, 22 bp, Artificial Sequence
  Chemcially synthesized probe for enteroviral RNA
ccgccacagacttgcgcattac

SEQ ID NO: 36 - DNA, 26 bp, Artificial Sequence
  Chemically synthesized probe for enteroviral RNA
tccgccacagacttgcgcattacgac

SEQ ID NO: 37 - DNA, 26 bp, Artificial Sequence
  Chemically synthesized probe for enteroviral RNA
ttccgccacagacttgcgcattacga

SEQ ID NO: 38 - DNA, 23 bp, Artificial Sequence
  Chemically synthesized probe for enteroviral RNA
tccgctgcagagttgcccgttac

SEQ ID NO: 39 - DNA, 23 bp, Artificial Sequence
  Chemically synthesized probe for enteroviral RNA
cgctgcagagttgcccgttacga

SEQ ID NO: 40 - DNA, 21 bp, Artificial Sequence
  Chemically synthesized probe for enteroviral RNA
cgctgcagagttgcccgttac

SEQ ID NO: 41 - DNA, 23 bp, Artificial Sequence
  Chemcially synthesized probe for enteroviral RNA
ccgctgcagagttgcccgttacg

SEQ ID NO: 42 - DNA, 24 bp, Artificial Sequence
  Chemically synthesized probe for enteroviral RNA
ccgctgcagagttgcccgttacga

SEQ ID NO: 43 - DNA, 24 bp, Artificial Sequence
  Chemically synthesizec probe for enteroviral RNA
tccgctgcagagttgcccgttacg

SEQ ID NO: 44 - DNA, 22 bp, Artificial Sequence
  Chemically synthesized probe for enteroviral RNA
cgctgcagagttgcccgttacg

SEQ ID NO: 45 - DNA, 22 bp, Artificial Sequence
  Chemically synthesized probe for enteroviral RNA
ccgctgcagagttgcccgttac

SEQ ID NO: 46 - DNA, 26 bp, Artificial Sequence
  Chemically synthesized probe for enteroviral RNA
tccgctgcagagttgcccgttacgac

SEQ ID NO: 47 - DNA, 26 bp, Artificial Sequence
  Chemically synthesized probe for enteroviral RNA
ttccgctgcagagttgcccgttacga

SEQ ID NO: 48 - DNA, 23 bp, Artificial Sequence
  Chemcially synthesized probe for enteroviral RNA
tccgctgcrgagttrcccrttac

SEQ ID NO: 49 - DNA, 23 bp, Artificial Sequence
  Chemically synthesized probe for enteroviral RNA
cgctgcrgagttrcccrttacga

SEQ ID NO: 50 - DNA, 21 bp, Artificial Sequence
  Chemcially synthesized probe for enteroviral RNA
cgctgcrgagttrcccrttac

SEQ ID NO: 51 - DNA, 23 bp, Artificial Sequence
  Chemically synthesized probe for enteroviral RNA
ccgctgcrgagttrcccrttacg

SEQ ID NO: 52 - DNA, 24 bp, Artificial Sequence
  Chemically synthesized probe for enteroviral RNA
ccgctgcrgagttrcccrttacga

SEQ ID NO: 53 - DNA, 24 bp, Artificial Sequence
  Chemically synthesized probe for enteroviral RNA
tccgctgcrgagttrcccrttacg

SEQ ID NO: 54 - DNA, 22 bp, Artificial Sequence
  Chemically synthesized probe for enteroviral RNA
cgctgcrgagttrcccrttacg

SEQ ID NO: 55 - DNA, 22 bp, Artificial Sequence
  Chemically synthesized probe for enteroviral RNA
ccgctgcrgagttrcccrttac

SEQ ID NO: 56 - DNA, 26 bp, Artificial Sequence
  Chemically synthesized probe for enteroviral RNA
tccgctgcrgagttrcccrttacgac

SEQ ID NO: 57 - DNA, 26 bp, Artificial Sequence
  Chemically synthesized probe for enteroviral RNA
ttccgctgcrgagttrcccrttacga

(19) United States
(12) Patent Application Publication
(10) Pub. No.: US 2005/0239055 A1
(43) Pub. Date: Oct. 27, 2005

(54) Enterovirus primers and probes

(76) Inventor: Brian D. Mariani, Herndon, VA (US)

Correspondence Address: MCDERMOTT, WILL & EMERY, 600 13th Street, N.W., Washington, DC, 20005-3096, US

(21) Appl. No.: 10/829,474

(22) Filed: Apr. 22, 2004

(51) Int. Cl. C12Q 1/70; C12Q 1/68; C07H 21/04

(52) U.S. Cl. 435/5; 435/6; 536/23.72

(57) ABSTRACT

The present invention provides methods, primers and probes for the detection of enteroviral nucleic acids in biological fluids and tissue. In the methods of the invention, at least a portion of enteroviral nucleic acid present in a biological sample suspected of containing an enterovirus is amplified and the amplified enteroviral nucleic acid is then detected. Detection may be accomplished by conventional separation techniques such as gel electrophoresis or by hybridization of at least a portion of a nucleotide probe comprising a nucleotide sequence complementary to the amplified enteroviral nucleic acid. Preferably, enteroviral RNA is detected in a biological sample using real-time PCR techniques that can detect the increasing presence of an amplification product while amplification occurs.

### BACKGROUND OF THE INVENTION

1. Field of the Invention

The present invention relates to primers, probes and methods for detecting viral infection of biological tissues, and more particularly to primers, probes and methods for detecting enteroviral infection of biological fluids and tissues using the primers and probes.

2. Background

The enteroviruses are a heterogeneous group of nearly 70 human pathogens which are responsible for a broad spectrum of clinical diseases resulting in mild flu-like symptoms, or no symptoms at all in healthy individuals. They are among the most common human viruses circulating worldwide. Like other members of the picomavirus family, the enteroviruses are small (27 nm), single-stranded, nonenveloped RNA viruses of approximately 1.34 g/ml buoyant density. Enteroviruses (EV) distinguish themselves from rhinoviruses, another type of human picomavirus, by their stability in acid, by their fecal-oral route of passage and transmission, and by their strict summer peak of disease activity. The prototypic enteroviruses, the polioviruses, remain the most clinically significant of the enteroviruses worldwide, causing paralytic disease in 4 of every 1,000 school-age children in developing countries. In the United States, the polioviruses have been controlled with the introduction of vaccines in the late 1950's.

The nonpolio enteroviruses, however, are responsible for 5 to 10 million symptomatic infections each year. They are the most common etiologic agents of meningitis (75,000 cases per year) and of nonspecific febrile and exanthematous illnesses (5 million cases per year). They are also responsible for significant numbers of cases of myocarditis, hepatitis, pleurodynia, stomatitis, and neonatal sepsis. Recently identified nonpolio enterovirus serotypes cause hemorrhagic conjunctivitis and poliomyelitis. Several important diseases are suspected of having an enteroviral etiology without definitive proof; these include diabetes mellitus, dermatomyositis, congenital hydrocephalus, and amyotrophic lateral sclerosis. The enteroviruses cause infections which may persist for many years in immunocompromised individuals, often leading to death. Recently, a syndrome of late onset muscular atrophy has been reported in individuals who suffered paralytic poliomyelitis 20 to 40 years previously.

Beyond the obvious desire to determine the specific etiology of these diverse and important diseases, there are many reasons for seeking a rapid and accurate diagnostic test for the enteroviruses. It is often clinically impossible to distinguish enteroviral infections from those due to bacterial pathogens or other viruses, including herpes simplex, for which there are specific therapies. Although many enteroviral infections are self-limited and require no therapy, the fear that an illness may be bacterial or herpetic results in unnecessary hospitalization and antibiotic or antiviral treatment for thousands of enterovirus-infected patients each year.

In the newborn, enteroviruses can cause mild symptoms, and if it gains entry into the central nervous system (CNS) it can lead to aseptic (viral) meningitis. While most serotypes are not necessarily dangerous to the newborn, the symptoms of aseptic meningitis can be undistinguishable from those resulting from Herpes Simplex Virus (HSV) infection of the CNS. Thus, EV infection becomes the most significant infectious differential diagnosis where neonatal HSV is suspected.

Symptoms elicited by EV infection can overlap significantly with those of HSV, confounding a proper diagnosis. Although the symptoms are similar, the treatment course and potential outcome for the patient differ greatly, with HSV infection warranting immediate antiviral treatment since untreated HSV can lead to severe patient morbidity and mortality, whereas EV infection is self-limiting and usually non-threatening. It is therefore valuable to have a rapid assay to accurately document EV in clinical samples.

In this regard, testing for EV in cases of neonatal fever, irritability, seizure, lethargy, etc., is valuable, since ˜22-25% of patients with these general symptoms have tested positive for EV. By having the rapid documentation of EV infection in newborns where HSV was a distinct possibility, a greater confidence level can be reached compared to relying on the HSV-negative result alone, since a HSV false-negative is less likely when a common viral agent exhibiting the same set of symptoms is definitively found instead. With greater diagnostic confidence comes improved patient management, since the EV-positive neonate will spend less time in the NICU, consume less (if any) anti-viral medication, and have a shorter time to discharge, with less additional, potentially expensive diagnostic procedures.

The Nobel Prize in medicine and physiology was awarded to J. F. Enders, F. C. Robbins, and T. H. Weller in 1954 for their success in cultivating poliovirus in tissue culture, an accomplishment which paved the way for vaccine development and provided a means for laboratory testing for the polio and nonpolio enteroviruses. Since then, tissue culture continues to be the mainstay of the enteroviral diagnosis despite well-recognized limitations. Tissue culture is time-consuming and requires a high level of expertise. Of greater concern is the fact that certain of the enteroviruses will not grow in tissue culture, requiring inoculations into suckling mice for detection, a technique cumbersome enough to be omitted from almost all diagnostic laboratories. The sensitivity of routine tissue culture for the enteroviruses may be as low as 65 to 75%, and development of characteristic cytopathic effect may take too long to be of benefit to the patient. Cerebrospinal fluid (CSF) infections with the enteroviruses take a mean of 6.3 days in the laboratory for growth in culture, consistent with reported means to isolation from the CSF of 4.0 to 8.2 days. Other body sites may become positive sooner, but as meningitis is the most vexing of enteroviral infections for the clinician, CSF data are the most relevant. The use of additional cell lines improves the yield at the cost of increasing the labor and resource required. Although the specificity is high, the sensitivity of EV culture suffers due to the high percentage of “failures-to-grow” in clinically confirmed EV positive cases. Thus, a rapid, highly sensitive and specific assay for EV detection fills a gap in existing techniques for the confirmation of EV infection, and allowing the exclusion of other more pathogenic viral agents sharing the same general symptoms.

Immunodiagnostic techniques for the enteroviruses have been fraught with difficulties resulting from the extreme antigenic diversity among the serotypes. Although a common antigen may exist among the polioviruses and another among the coxsackievirus B types, checkerboard pools of antisera would be required to cover even the most common enteroviral serotypes responsible for human disease. Serologic testing suffers from the same lack of a ubiquitous enteroviral antigen as immunoassays do, requiring, in this case, pools of antigens for testing. Coxsackievirus type B immunoglobulin M serology has the most proven clinical application. It has been found to be advantageous because of shared antigen and early appearance of the immunoglobulin M class of antibodies. Immunoglobulin G serology for the enteroviruses is useful for epidemiologic studies, but of little benefit to the individual patient.

DNA and RNA probes have been used to detect enteroviruses. In Rotbart et al., J. Clin. Microbiol. 20: 1105-1108, (1984), three nucleotide hybridization probes derived from DNA clones of the poliovirus type 1 genome were used in dot hybridization experiments. The probes successfully detected members of each of the major enteroviral subgroups. In Rotbart et al., J. Clin. Microbiol 22: 220-224, (1985), cDNA probes derived from poliovirus 1 and coxsackievirus B3 were used to detect enteroviruses in cerebrospinal fluid reconstruction experiments where an array of enteroviruses were added to cerebrospinal fluid. The viruses were detected by a dot hybridization assay using cDNA probes. Although cDNA probes have been able to detect enteroviruses in cerebrospinal fluid reconstructions, in clinical tests the probes were relatively insensitive in detecting enteroviral infection, Rotbart and Levin, Chapter 15, “Progress Toward the Development of a Pan-Enteroviral Nucleic Acid Probe”, in DNA Probes for Infectious Diseases, pp. 193-209, 197. In the clinical tests, two thirds of cerebrospinal test fluids that proved positive with tissue culture were missed by the cDNA probes. Single stranded RNA probes (Rotbart et al., Molecular and Cellular Probes 2: 65-73, (1988) can be several times more sensitive than cDNA probes, however, even this improved sensitivity may be too little to routinely detect enteroviruses in cerebrospinal fluid. It is estimated that cerebrospinal fluid from patients with aseptic meningitis due to human enterovirus contains 10-10³ virions per milliliter. The sensitivity of the RNA probes approached this level; nevertheless, the low levels of virus in body fluids preclude the reliable use of the probes for diagnosing picornaviral infection on a routine basis.

There is thus a great need for sensitive methods for detecting enteroviruses in biological fluids and tissues that can be applied to the small amounts of virus often present and that can be quickly performed so that timely diagnosis of infection can be made.

All articles and patent documents cited herein are expressly incorporated by reference for their entirety for all purposes, particularly, Byington et al., Pediatrics, March 1999, 103(3); Pediatr Ann., 2002, November; 31(11): 726-32; Pfaller et al., Emerg Infect Dis, 2001, 7(2), 1-11; Ramers et al., JAMA, 2000, May 24-31; 283(20): 2680-5; Verstrepen et al., J Clin Virol. 2002 Jul. 25, Suppl 1:S39-43; Kockx et al., J Clin Microbiol 2001 39:4093-6.

### SUMMARY OF THE INVENTION

One aspect of the invention relates to an isolated oligonucleotide of the sequence SEQ ID NO: 1. One embodiment of this aspect of the invention relates to an isolated oligonucleotide that hybridizes the complement of SEQ ID NO: 1 under stringent conditions and is capable of amplifying reverse transcribed enteroviral RNA when used in conjunction with SEQ ID NO: 2 in an polymerase chain reaction. Another embodiment of this aspect of the invention relates to an isolated oligonucleotide of the sequence of SEQ ID NO: 1, wherein about one to about three nucleotides are added or removed from the ′5 end and/or about one to about three nucleotides are added or removed from the 3′ end, respectively.

Another aspect of the invention relates to an isolated oligonucleotide of the sequence SEQ ID NO: 2. One embodiment of this aspect of the invention relates to an isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction. Another embodiment relates to an isolated oligonucleotide of the sequence of SEQ ID NO: 2, wherein about one to about three nucleotides are added or removed from the ′5 end and/or about one to about three nucleotides are added or removed from the 3′ end, respectively.

Another aspect of the invention relates to an isolated oligonucleotide having the sequence of SEQ ID NO: 3 or a sequence wherein about one to about three nucleotides are added or removed from the ′5 end and/or about one to about three nucleotides are added or removed from the 3′ end of SEQ ID NO: 3.

Another aspect of the invention relates to kit for detecting enteroviral RNA comprising a first isolated oligonucleotide of SEQ ID NO: 1 and a second oligonucleotide of SEQ ID NO: 2 or an oligonucleotide substantially identical thereto.

Another aspect of the invention relates to kit for detecting enteroviral RNA comprising a first isolated oligonucleotide of SEQ ID NO: 2 and a second oligonucleotide of SEQ ID NO: 1 or an oligonucleotide substantially identical thereto.

Another aspect of the invention relates to kit for detecting enteroviral RNA comprising a first isolated oligonucleotide of SEQ ID NO: 1 and a second oligonucleotide of SEQ ID NO: 2.

Another aspect of the invention relates to a kit for detecting enteroviral RNA comprising a first oligonucleotide selected from the group consisting of: an isolated oligonucleotide of the sequence SEQ ID NO: 1; an isolated oligonucleotide that hybridizes the complement of SEQ ID NO: 1 under stringent conditions and is capable of amplifying reverse transcribed enteroviral RNA when used in conjunction with SEQ ID NO: 2 in an polymerase chain reaction; and an isolated oligonucleotide of the sequence of SEQ ID NO: 1, wherein about one to about three nucleotides are added or removed from the ′5 end and/or about one to about three nucleotides are added or removed from the 3′ end, respectively; and a second oligonucleotide selected from the group consisting of an isolated oligonucleotide of the sequence SEQ ID NO: 2; an isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction; and an isolated oligonucleotide of the sequence of SEQ ID NO: 2, wherein about one to about three nucleotides are added or removed from the ′5 end and/or about one to about three nucleotides are added or removed from the 3′ end, respectively.

Another aspect of the invention relates to a method of detecting the presence of enteroviral RNA in a biological sample comprising: obtaining a biological sample from an organism; isolating nucleic acids from tha sample; performing a polymerase chain reaction on tha isolated nucleic acids using a first isolated oligonucleotide selected from the group consisting of: an isolated oligonucleotide of the sequence SEQ ID NO: 1; an isolated oligonucleotide that hybridizes the complement of SEQ ID NO: 1 under stringent conditions and is capable of amplifying reverse transcribed enteroviral RNA when used in conjunction with SEQ ID NO: 2 in an polymerase chain reaction; and an isolated oligonucleotide of the sequence of SEQ ID NO: 1, wherein about one to about three nucleotides are added or removed from the ′5 end and/or about one to about three nucleotides are added or removed from the 3′ end, respectively; and a second oligonucleotide selected from the group consisting of an isolated oligonucleotide of the sequence SEQ ID NO: 2; an isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction; and an isolated oligonucleotide of the sequence of SEQ ID NO: 2, wherein about one to about three nucleotides are added or removed from the ′5 end and/or about one to about three nucleotides are added or removed from the 3′ end, respectively, correlating a presence of an amplification product from tha polymerase chain reaction with the presence of enteroviral RNA is tha sample.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention. Accordingly, the drawings and description are to be regarded as illustrative in nature, and not as restrictive.

### BRIEF DESCRIPTION OF THE DRAWINGS

FIG. 1 EV Dilution Series: Detection of Coxsackie A16 virus at 5,000, 1,000, 100, 10 genomes per reaction (ROX).

FIG. 2. Detection of 10 copies of Coxsackie A16 in 16 parallel reactions (ROX).

FIG. 3. Detection of several dominant EV serotypes (˜5,000 virus/rxn) either pure or in a background of human RNA (ROX).

FIG. 4. Detection of EV in archived CSF samples with and without a human RNA “spike” (ROX). Results correlate with “nested” RT-PCR results using PAGE.

FIG. 5. Detection of human beta-actin mRNA in archived CSF samples (from above, #4). Patient-derived internal positive control signal was detected in all samples (FAM).

FIG. 6. Additional set of archived CSF samples (positives and negatives) showing EV detection (ROX).

FIG. 7. Same samples as above (#6) showing detection of beta-actin internal control (FAM).

FIG. 8. Detection of EV in archived plasma samples (positives & negatives) (ROX).

FIG. 9. Detection of beta-actin internal positive control in same plasma samples as above (#8) (FAM).

FIG. 10. Detection of EV in a blood from clinically determined “high” and “low” titer” infections (each run in duplicate) (ROX).

FIG. 11. Detection of beta-actin internal positive control in the samples shown above (#10) (FAM).

FIG. 12. EV Real-time RT-PCR assay performed on known negative CSF samples (ROX).

FIG. 13. Detection of beta-actin internal positive control in above negative samples (FAM).

### DETAILED DESCRIPTION OF THE INVENTION

The present invention provides methods, primers and probes for the detection of enteroviral infections in biological fluids and tissue. In the methods of the invention, at least a portion of enteroviral nucleic acid present in a biological sample suspected of containing an enterovirus is amplified (i.e. multiple copies of the nucleic acid are made) and the amplified enteroviral nucleic acid is then detected. Detection may be accomplished by conventional separation techniques such as gel electrophoresis or by hybridization of at least a portion of a nucleotide probe comprising a nucleotide sequence complementary to the amplified enteroviral nucleic acid. The amplified enteroviral nucleic may also be detected by any suitable combination of detection techniques such as gel electrophoresis followed by hybridization with a nucleic acid probe. Preferably, enteroviral RNA is detected in a biological sample using real-time PCR techniques that can detect the increasing presence of an amplification product while amplification occurs.

“Nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

One aspect of the invention relates to oligonucleotide primers capable of acting as forward primers in a polymerase chain reaction (PCR) for amplifying enteroviral RNA. Preferably, the forward primer has the sequence: 5′-CCCCTGAATGCGGCTAATC-3′ (SEQ ID NO: 1).

Another aspect of the invention relates to oligonucleotide primers capable of acting as reverse primers in a PCR reaction for amplifying enteroviral RNA. Preferably, the reverse primer has the sequence: 5′-AAGGAAACACGGACACCCAA-3′ (SEQ ID NO: 2).

The term “oligonucleotide” refers to a molecule comprised of two or more deoxyribonucleotides or ribonucleotides, such as primers, probes, nucleic acid fragments to be detected, and nucleic acid controls. The exact size of an oligonucleotide depends on many factors and the ultimate function or use of the oligonucleotide.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The appropriate length of a primer depends on the intended use of the primer but typically ranges from about 10 to about 30 nucleotides. Short primer molecules generally require cooler temperatures to form sufficiently stable hybrid complexes with the template. A primer need not reflect the exact sequence of the template but must be sufficiently complementary to specifically hybridize with a template. When primer pairs are referred to herein, the pair is meant to include one forward primer which is capable of hybridizing to the sense strand of a double-stranded target nucleic acid (the “sense primer”) and one reverse primer which is capable of hybridizing to the antisense strand of a double-stranded target nucleic acid (the “antisense primer”).

Another aspect of the invention relates to oligonucleotides capable of acting as probe for enteroviral RNA. Preferably, the probe has the sequence: 5′-TCCGCCACAGACTTGCGCATTACGA-3′ (SEQ ID NO: 3).

Another aspect of the invention relates to oligonucleotides capable of acting as probe for enteroviral RNA. Preferably, the probe has the sequence: 5′-TCCGCTGCAGAGTTGCCCGTTACGA-3′ (SEQ ID NO: 4).

TABLE 1

Selected primer and probe sequences for RT-PCR detection
of enterovirus

| Sequence | | | | GC | Tₘ | Detection |
|---|---|---|---|---|---|---|
| Name | Nucleotide Sequence 5′to 3′ | Position | Length | (%) | (° C) | System* |
| EV F | 5′-CCC CTG AAT GCG GCT | 454-472 | 19- | 58% | 71.2 | |
| (forward) | AAT C-3′ | 5′-UTR | mer | | | |
| (SEQ ID | | | | | | |
| NO:1) | | | | | | |
| EV R | 5-AAG GAA ACA CGG ACA | 549-568 | 20- | 50% | 71.1 | |
| (reverse) | CCC AA-3′ | 5′-UTR | mer | | | |
| (SEQ ID | | | | | | |
| NO:2) | | | | | | |
| EV | 5′- (Reporter)-(TCC GCC | 518 of 5′- | 25- | 56% | 73.0 | Hydrolysis |
| probeA | ACA GAC TTG CGC ATT | UTR | mer | | | probe |
| (SEQ ID | ACG A) - (Quencher) - 3′ | | | | | |
| NO:3) | | | | | | |
| EV | 5′- (Reporter)-(TCC GCT | 518 of 5′- | 25- | 60% | 74.1 | Hydrolysis |
| probeB | GCA GAG TTG CCC GTT | UTR | mer | | | probe |
| (SEQ ID | ACG A) -(Quencher) - 3′ | | | | | |
| NO:4) | | | | | | |

Another aspect of the invention relates to oligonucleotides capable of acting as probe for enteroviral RNA. Preferably, the probe has the sequence: 5′-TCCGCTGC(G/A)GAGTT(A/G)CCC(A/G)TTACGA-3′ (SEQ ID NO: 5). “G/A” refers to the presence of either a G or A.

“Probe” refers to an oligonucleotide which binds through complementary base pairing to a sub-sequence of a target nucleic acid. A primer may be a probe. It will be understood by one of skill in the art that probes will typically substantially bind target sequences lacking complete complementarity with the probe sequence depending upon the stringency of the hybridization conditions. The probes are typically directly labeled (e.g., with isotopes or fluorescent moieties) or indirectly labeled such as with biotin to which a streptavidin complex may later bind. By assaying for the presence or absence of the probe, one can detect the presence or absence of the target, by Southern blot for example. Preferably, the target is an amplification product generated by PCR amplification of enteroviral nucleic acids using primers SEQ ID NOs: 1 and 2, or oligonucleotides substantially identical thereto, respectively. More, preferably, the probes are fluorescently labeled and are capable of acting as a hydrolysis or Taqman® probes in a real-time PCR reaction to amplify enteroviral nucleic acids. The real-time PCR method uses a dual labelled fluorogenic oligonucleotide probe (i.e., the hydrolysis or TaqMan® probe) that anneals specifically within the template amplicon spanning the forward (e.g. SEQ ID NO: 1) and reverse primers (e.g. SEQ ID NO: 2). Preferably, a fluorescent reporter molecule attached to 5′-position that fluoresces when released from probe by hydrolytic activity of DNA polymerase. Preferably, a quencher is attached to the 3′ end of the probe. A quencher is a fluorescent molecule that quenches (absorbs) the fluorescence emission from the Reporter only when Reporter is attached to the probe before hydrolysis. Preferably, laser stimulation within the capped wells containing the reaction mixture causes emission of a 3′ quencher dye (TAMRA) until the probe is cleaved by the 5′ to 3′ nuclease activity of the DNA polymerase during PCR extension, causing release of a 5′ reporter dye (6FAM). Preferably, production of an amplicon causes emission of a fluorescent signal that is detected by a CCD (charge-coupled device) detection camera or other light capturing device, and the amount of signal produced at a threshold cycle within the purely exponential phase of the PCR reaction, reflects the starting copy number of the target sequence being amplified.

The invention also includes oligonucleotides substantially identical to SEQ ID NOs: 1-5. For example, oligonucleotide sequences substantially identical to SEQ ID NOs: 1-5 may have several nucleotides added to or removed from their 5′ ends or several nucleotides added to or removed from their 3′ ends. “Several nucleotides” in this context refers to about 3 nucleotides, or preferably about two nucleotides or more preferably about one nucleotide. The person of skill in the art will recognize that when adding nucleotides to the 5′ and/or 3′ ends SEQ ID NOs: 1-5, the identity of those nucleotides may be dictated by the sequence of the reverse transcribed enteroviral RNA(cDNA) to be amplified. Preferably, the template is the 5′ untranslated region of the enteroviral RNA genome. However, the skilled artisan may also wish to add overhangs to the 5′ end of the forward primer or 5′ end of the reverse primer (giving a 3′ sticky end on the amplicon). Such overhangs may include restriction enzyme sites useful in providing sticky ends to facilitate subcloning of the amplification product, for example.

Primers and probes of the invention exhibit an absence of hybridization to sequences contained in human RNA and DNA. This may be confirmed theoretically by BLAST analysis (NCBI), and empirically by testing selected primer sets against human total nucleic acid under both RT and PCR conditions. Additionally, the claims probes and primers lack cross reactivity against other non-enteroviral genomes that could be present in clinical samples. This may also be confirmed theoretically in a BLAST search, and empirically using HSV1, HSV2, CMV, EBV, HHV6, HIV, HCV, HBV, parvovirus genomic material.

Primers and probes substantially identical to SEQ ID NOs: 1-5 must be reactive with the dominant EV serotypes circulating within the geographic region spanning the target patient population. Annually, the CDC posts the dominant EV serotypes circulating in the U.S. Of the ˜66 serotypes, about on 12 to 15 (depending on year) make up 95% of the transmittable virus. Detection of these serotypes is determined by using EV serotype stocks obtained from ATCC (see below). Each serotype is tested for detection using dilutions to 1:10,000 and 1:100,000 of stock culture. Detection of at least the 1:10,000 dilution of each serotype stock must be achieved to ensure clinical sensitivity and specificity.

For example, one of skill in the art would envisage a genus of sequences substantially identical to SEQ ID NO: 1 wherein about one to about three nucleotides are added or removed from the ′5 end and/or about one to about three nucleotides are added or removed from the 3′ end, respectively, to include but not be limited to the following exemplary species:

| | | |
|---|---|---|
| Seq. | | |
| ID No. | Sequence Substantially Identical to SEQ ID NO: 1 | Notes |

| | | |
|---|---|---|
| 6 | 5′-CCCCTGAATGCGGCTAA-3′ | 2 nt removed from ′3 end |
| 7 | 5′-CCTGAATGCGGCTAATC-3′ | 2 nt removed from ′5 end |
| 8 | 5′-CCTGAATGCGGCTAA-3′ | 2 nt removed from ′5 end and 2 nt |
| | | removed from ′3 end |
| 9 | 5′-CCCTGAATGCGGCTAAT-3′ | 1 nt removed from ′5 end and 1 nt |
| | | removed from ′3 end |
| 10 | 5′-CCCTGAATGCGGCTAATC-3′ | 1 nt removed from ′5 end |
| 11 | 5′-CCCCTGAATGCGGCTAAT-3′ | 1 ntremoved from ′3 end |
| 12 | 5′-CCTGAATGCGGCTAAT-3′ | 2 nt removed from ′5 end and 1 ut |
| | | removed from ′3 end |
| 13 | 5′-CCCTGAATGCGGCTAAT-3′ | 1 nt removed from ′5 end and 2 nt |
| | | removed from ′3 end |
| 14 | 5′-CCCCTGAATGCGGCTAATCT-3′ | 2 nt added to ′3 end |
| 15 | 5′-GCCCCTGAATGCGGCTAATC-3′ | 2 nt added to ′5 end |
| 16 | 5′-G\hat{ }AATTCCCCCTGAATGCGGCTAATC-3′ | EcoRI site added to 5′ end |

Additionally, one of skill in the art would envisage a genus of sequences substantially identical to SEQ ID NO: 2 wherein about one to about three nucleotides are added or removed from the ′5 end and/or about one to about three nucleotides are added or removed from the 3′ end, respectively, to include but not be limited to the following exemplary species:

| Seq. | | |
|---|---|---|
| ID No. | Sequence Substantially Identical to SEQ ID NO: 2 | Notes |
| 17 | 5′- AAGGAAACACGGACACCC-3′ | 2 nt removed from ′3 end |
| 18 | 5′- GGAAACACGGACACCCAA-3′ | 2 nt removed from ′5 end |
| 19 | 5′- GGAAACACGGACACCC-3′ | 2 nt removed from ′5 end and 2 |
| | | nt removed from ′3 end |
| 20 | 5′- AGGAAACACGGACACCCA-3′ | 1 nt removed from ′5 end and 1 |
| | | nt removed from ′3 end |
| 21 | 5′- AGGAAACACGGACACCCAA-3′ | 1 nt removed from ′5 end |
| 22 | 5′- AAGGAAACACGGACACCCA-3′ | 1 nt removed from ′3 end |
| 23 | 5′- GGAAACACGGACACCCA-3′ | 2 nt removed from ′5 end and 1 |
| | | nt removed from ′3 end |
| 24 | 5′- AGGAAACACGGACACCC-3′ | 1 nt removed from ′5 end and 2 |
| | | nt removed from ′3 end |
| 25 | 5′- AAGGAAACACGGACACCCAAA-3′ | 1 nt added to ′3 end |
| 26 | 5′- AAAGGAAACACGGACACCCAA-3′ | 1 nt added to ′Send |
| 27 | 5′- G\hat{ }AATTCAAGGAAACACGGACACCCAA-3′ | EcoRI site added to 5′ end |

One of skill in the art would envisage a genus of sequences substantially identical to SEQ ID NO: 3 wherein about one to about three nucleotides are added or removed from the ′5 end and/or about one to about three nucleotides are added or removed from the 3′end, respectively, to include but not be limited to the following exemplary species:

| Seq. | | |
|---|---|---|
| ID No. | Sequence Substantially Identical to SEQ ID NO: 3 | Notes |
| 28 | 5′- TCCGCCACAGACTTGCGCATTAC-3′ | 2 nt removed from ′3 end |
| 29 | 5′- CGCCACAGACTTGCGCATTACGA-3′ | 2 nt removed from ′5 end |
| 30 | 5′- CGCCACAGACTTGCGCATTAC-3′ | 2 nt removed from ′5 end and 2 |
| | | nt removed from ′3 end |
| 31 | 5′- CCGCCACAGACTTGCGCATTACG-3′ | 1 nt removed from ′5 end and 1 |
| | | nt removed from ′3 end |
| 32 | 5′- CCGCCACAGACTTGCGCATTACGA-3′ | 1 nt removed from ′5 end |
| 33 | 5′- TCCGCCACAGACTTGCGCATTACG-3′ | 1 nt removed from ′3 end |
| 34 | 5′- CGCCACAGACTTGCGCATTACG-3′ | 2 nt removed from ′5 end and 1 |
| | | nt removed from ′3 end |
| 35 | 5′- CCGCCACAGACTTGCGCATTAC-3′ | 1 nt removed from ′5 end and 2 |
| | | nt removed from ′3 end |
| 36 | 5′- TCCGCCACAGACTTGCGCATTACGAC-3′ | 1 nt added to ′3 end |
| 37 | 5′- TTCCGCCACAGACTTGCGCATTACGA-3′ | nt added to ′Send |

One of skill in the art would envisage a genus of sequences substantially identical to SEQ ID NO: 4 wherein about one to about three nucleotides are added or removed from the ′5 end and/or about one to about three nucleotides are added or removed from the 3′ end, respectively, to include but not be limited to the following exemplary species:

| | | |
|---|---|---|
| Seq. | | |
| ID No. | Sequence Substantially Identical to SEQ ID NO: 4 | Notes |

| | | |
|---|---|---|
| 38 | 5′-TCCGCTGCAGAGTTGCCCGTTAC-3′ | 2 nt removed from ′3 end |
| 39 | 5′-CGCTGCAGAGTTGCCCGTTACGA-3′ | 2 nt removed from ′5 end |
| 40 | 5 ′-CGCTGCAGAGTTGCCCGTTAC-3′ | 2 nt removed from ′5 end and 2 |
| | | nt removed from ′3 end |
| 41 | 5′-CCGCTGCAGAGTTGCCCGTTACG-3′ | 1 nt removed from ′5 end and 1 |
| | | nt removed from ′3 end |
| 42 | 5′-CCGCTGCAGAGTTGCCCGTTACGA-3′ | 1 nt removed from ′5 end |
| 43 | 5′-TCCGCTGCAGAGTTGCCCGTTACG-3′ | 1 nt removed from ′3 end |
| 44 | 5′-CGCTGCAGAGTTGCCCGTTACG-3′ | 2 nt removed from ′5 end and 1 |
| | | nt removed from ′3 end |
| 45 | 5′-CCGCTGCAGAGTTGCCCGTTAC-3′ | 1 nt removed from ′5 end and 2 |
| | | nt removed from ′3 end |
| 46 | 5′-TCCGCTGCAGAGTTGCCCGTTACGAC-3′ | 1 nt added to ′3 end |
| 47 | 5′-TTCCGCTGCAGAGTTGCCCGTTACGA-3′ | 1 nt added to ′5 end |

Finally, one of skill in the art would envisage a genus of sequences substantially identical to SEQ ID NO: 5 wherein about one to about three nucleotides are added or removed from the ′5 end and/or about one to about three nucleotides are added or removed from the 3′ end, respectively, to include but not be limited to the following exemplary species.

| Seq. | | |
|---|---|---|
| ID No. | Sequence Substantially Identical to SEQ ID NO: 5 | Notes |
| 48 | 5′-TCCGCTGC(G/A)GAGTT(A/G)CCC(A/G)TTAC-3′ | 2 nt removed from ′3 |
| | | end |
| 49 | 5′-CGCTGC(G/A)GAGTT(A/G)CCC(A/G)TTACGA-3′ | 2 nt removed from ′5 |
| | | end |
| 50 | 5′-CGCTGC(G/A)GAGTT(A/G)CCC(A/G)TTAC-3′ | 2 nt removed from ′5 |
| | | end and 2 nt removed |
| | | from ′3 end |
| 51 | 5 ′-CCGCTGC(G/A)GAGTT(A/G)CCC(A/G)TTACG-3′ | 1 nt removed from ′5 |
| | | end and 1 nt removed |
| | | from ′3 end |
| 52 | 5′-CCGCTGC(G/A)GAGTT(A/G)CCC(A/G)TTACGA-3′ | 1 nt removed from ′5 |
| | | end |
| 53 | 5′-TCCGCTGC(G/A)GAGTT(A/G)CCC(A/G)TTACG-3′ | 1 nt removed from ′3 |
| | | end |
| 54 | 5′-CGCTGC(G/A)GAGTT(A/G)CCC(A/G)TTACG-3′ | 2 nt removed from ′5 |
| | | end and 1 nt removed |
| | | from ′3 end |
| 55 | 5′-CCGCTGC(G/A)GAGTT(A/G)CCC(A/G)TTAC-3′ | 1 nt removed from ′5 |
| | | end and 2 nt removed |
| | | from ′3 end |
| 56 | 5′-TCCGCTGC(G/A)GAGTT(A/G)CCC(A/G)TTACGAC- | 1 nt added to ′3 end |
| | 3′ | |
| 57 | 5′-TTCCGCTGC(G/A)GAGTT(A/G)CCC(A/G)TTACGA- | 1 nt added to ′5 end |
| | 3′ | |

The skilled artisan will also appreciate that oligonucleotide sequences substantially identical to SEQ ID NOs: 1-5 may differ from SEQ ID NOs: 1-5, respectively, with respect to the identity of at least one nucleotide base. However, all oligonucleotides sequences substantially identical to SEQ ID NOs: 1-5 will hybridize under stringent conditions (as defined herein) to all or a portion of the complements of SEQ ID NOs: 1-5 (i.e., target sequences), respectively. The terms “hybridize(s) specifically” or “specifically hybridize(s)” refer to complementary hybridization between an oligonucleotide (e.g., a primer or labeled probe) and a target sequence. The term specifically embraces minor mismatches that can be accommodated by reducing the stringency of the hybridization media to achieve the desired priming for the PCR polymerases or detection of hybridization signal.

Under stringent hybridization conditions, only highly complementary, i.e., substantially identical nucleic acid sequences, hybridize. Preferably, such conditions prevent hybridization of nucleic acids having 3 or more mismatches out of 20 contiguous nucleotides, more preferably 2 or more mismatches out of 20 contiguous nucleotides, most preferably one or more mismatch out of 20 contiguous nucleotides. The hybridizing portion of the hybridizing nucleic acid is at least about 90%, preferably at least about 95%, or most preferably about at least about 98%, identical to the sequence of a target sequence, or its complement.

Hybridization of a nucleic acid to a nucleic acid sample under stringent conditions is defined below. Nucleic acid duplex or hybrid stability is expressed as a melting temperature (Tₘ), which is the temperature at which the probe dissociates from the target DNA. This melting temperature is used to define the required stringency conditions. If sequences are to be identified that are substantially identical to the probe, rather than identical, then it is useful to first establish the lowest temperature at which only homologous hybridization occurs with a particular concentration of salt (e.g. SSC or SSPE). Then assuming that 1% mismatching results in a 1° C. decrease in Tₘ, the temperature of the final wash in the hybridization reaction is reduced accordingly (for example, if sequences having >95% identity with the probe are sought, the final wash temperature is decrease by 5° C.). In practice, the change in Tₘ can be between 0.5° C. and 1.5° C. per 1% mismatch.

Stringent conditions involve hybridizing at 68° C. in 5×SSC/5× Denhart's solution/1.0% SDS, and washing in 0.2×SSC/0.1% SDS at room temperature. Moderately stringent conditions include washing in 3×SSC at 42° C. The parameters of salt concentration and temperature may be varied to achieve optimal level of identity between the primer and the target nucleic acid. Additional guidance regarding such conditions is readily available in the art, for example, Sambrook, Fischer and Maniatis, Molecular Cloning, a laboratory manual, (2nd ed.), Cold Spring Harbor Laboratory Press, New York, (1989) and F. M. Ausubel et al eds., Current Protocols in Molecular Biology, John Wiley and Sons (1994).

The probes and primers disclosed herein to detect a broad range of enteroviruses. Preferably, the oligonucleotides disclosed herein the presence of the following enteroviruses in a biological sample: Human Coxsackievirus A16, Human Coxsackievirus A3, Human Coxsackievirus A21, Human Coxsackievirus B1, Human enterovirus 70, Human enterovirus 71, Human echovirus 11, Human echovirus 14, Human echovirus 30, Human echovirus 6, Human echovirus 9, Human Coxsackievirus A9, Human Coxsackievirus B2, Human Coxsackievirus B3, Human Coxsackievirus B4, Human Coxsackievirus B5, Human echovirus 13, Human echovirus 18, Human echovirus 25, and Human echovirus 4.

Another aspect of the invention relates to a method of detecting enteroviral RNA by using SEQ ID NOs: 1 and 2; or oligonucleotides substantially identical thereto, in a polymerase chain reaction performed on a biological sample.

Another aspect of the invention relates to a kit for detecting enteroviral RNA having SEQ ID NOs: 1 and 2 or oligonucleotides substantially identical thereto. One embodiment of this aspect of the invention utilizes real-time PCR and includes at least on probe sequence selected from SEQ ID NO: 3, SEQ ID NO: 4, and SEQ ID NO: 5, or oligonucleotides substantially identical thereto.

The present methods and oligonucleotides can be applied to any type of biological sample that is suspected of containing enteroviral RNA. The term “biological sample” refers to a sample comprising any biological material (e.g., biological fluids or tissues) containing nucleic acids. Biological samples can include tissue samples, whole blood or serum, sputum, stool, urine, semen, pericardial fluid, nasopharyngeal/throat swabs, cerebrospinal fluid (CSF), amniotic fluid and the like. Preferably, the biological sample is CSF or blood serum. Tissues may, for example, be surgically resected from a patient in the form of a biopsy or autopsy tissue sample. Preferably, at least about 50 mg of tissue is resected. Preferably, all biological samples are transported at room temperature for overnight shipping and immediate processing. All bodily fluid biological samples (other than whole blood) may be stored frozen if processed at a later time.

In one embodiment where blood is the biological sample, peripheral blood is collected in an EDTA blood tube (1-3 ml). For neonates, 0.5-1.0 ml peripheral or heel-stick blood is preferable. In special cases where blood is limiting (premature births), the volume is preferably not less than about 0.2 ml.

In another embodiment, about 1.0 ml volume of CSF is collected in a sterile collection tube as a biological sample. For newborns, not less than 0.2-0.5 ml is preferable.

In another embodiment, about 1.0 ml of pericardial fluid is collected as a biological sample.

In another embodiment, about 2 to 3.0 ml of amniotic fluid is collected as a biological sample. Cellular material may be removed by centrifugation.

In another embodiment nasopharyngeal and throat swabs in the form of Dacron collection swab in about 3.0 ml of M4 transport media are used as a biological sample.

To amplify a target nucleic acid sequence in a biological sample by PCR, the sequence must be accessible to the components of the amplification system. In general, this accessibility is ensured by isolating the nucleic acids from the sample.

Preferably, the methods of the invention are performed with total RNA isolated from the biological sample, as the starting material. A variety of techniques for extracting nucleic acids, in particular ribonucleic acids, from biological samples are known in the art. Alternatively, if the sample is fairly readily disruptable, the nucleic acid may not need to be purified prior to amplification by the PCR technique, i.e., if the sample is comprised of cells, particularly peripheral blood lymphocytes or monocytes, lysis and dispersion of the intracellular components may be accomplished merely by suspending the cells in hypotonic buffer.

If it is not possible to extract RNA from the tissue sample soon after its resection, the sample may be fixed or frozen. RNA extracted and isolated from frozen or fresh samples of resected tissue is extracted by any method known in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of RNA during the extraction process.

Alternatively, tissue obtained from the patient may be fixed, preferably by formalin (formaldehyde) or gluteraldehyde treatment, for example. Biological samples fixed by alcohol immersion are also contemplated in the present invention. Fixed biological samples are often dehydrated and embedded in paraffin or other solid supports known to those of skill in the art. Such solid supports are envisioned to be removable with organic solvents, allowing for subsequent rehydration of preserved tissue. Fixed and paraffin-embedded (FPE) tissue sample as described herein refers to storable or archival tissue samples.

RNA may be extracted from a frozen or FPE sample by any of the methods as described in U.S. Pat. No. 6,428,963, which is hereby incorporated by reference in its entirety. In one embodiment of the invention, RNA is isolated from an archival pathological sample or biopsy which is first deparaffinized. An exemplary deparaffinization method involves washing the paraffinized sample with an organic solvent, such as xylene. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration. Alternatively, the sample is simultaneously deparaffinized and rehydrated.

Once the sample is reyhdrated, RNA is extracted and isolated from the rehydrated tissue. Deparaffinized samples can be homogenized using mechanical, sonic or other means of homogenization, e.g. by laser microdisection. In one embodiment, rehydrated samples are homogenized in a solution comprising a chaotropic agent, such as guanidinium thiocyanate (also sold as guanidinium isothiocyanate).

Chaotropic agents include but not limited to: guanidinium compounds, urea, formamide, potassium iodiode, potassium thiocyantate and similar compounds. The preferred chaotropic agent for the methods of the invention is a guanidinium compound, such as guanidinium isothiocyanate (also sold as guanidinium thiocyanate) and guanidinium hydrochloride. Many anionic counterions are useful, and one of skill in the art can prepare many guanidinium salts with such appropriate anions. The effective concentration of guanidinium solution used in the invention generally has a concentration in the range of about 1 to about 5M with a preferred value of about 4M. If RNA is already in solution, the guanidinium solution may be of higher concentration such that the final concentration achieved in the sample is in the range of about 1 to about 5M. The guanidinium solution also is preferably buffered to a pH of about 3 to about 6, more preferably about 4, with a suitable biochemical buffer such as Tris-Cl. The chaotropic solution may also contain reducing agents, such as dithiothreitol (DTT), (β-mercaptoethanol; BME); and combinations thereof. The chaotropic solution may also contain RNAse inhibitors.

RNA is then recovered from the solution by, for example, phenol chloroform extraction, ion exchange chromatography or size-exclusion chromatography. RNA may then be further purified using the techniques of extraction, electrophoresis, chromatography, precipitation or other suitable techniques.

Once total RNA has been isolated from a biological sample, the RNA is then transcribed into cDNA with reverse transcriptase. Reverse transcription (RT) of total RNA isolated from a biological sample is may be converted to cDNA using random hexamers, for example. Preferably, SEQ ID NO: 2 or oligonucleotides substantially identical thereto are used to primer reverse transcriptase. This step can be performed as the first round of amplification or can be performed separately. The cDNA is then amplified, preferably using the polymerase chain reaction (PCR) method, as disclosed in U.S. Pat. Nos. 4,683,195 and 4,683,202, the disclosures of which are specifically incorporated as if fully set forth herein. RT conditions have been previously described for frozen tissue (Horikoshi et al., 1992). Controls omitting the reverse transcriptase (No-RT) may also be prepared.

The amplification of enteroviral cDNA reverse transcribed from total RNA isolated from a fresh, frozen or fixed biological sample is preferably carried out using polymerase chain reaction (PCR) methods common in the art. The first step of each cycle of the PCR involves the separation of the nucleic acid duplex formed by the primer extension. Once the strands are separated, the next step in PCR involves hybridizing the separated strands with primers that flank the target sequence e.g. SEQ ID NOs: 1 and 2. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid. Strand separation is achieved by heating the reaction to a sufficiently high temperature for a sufficient time to cause the denaturation of the duplex but not to cause an irreversible denaturation of the polymerase (see U.S. Pat. No. 4,965,188). Template-dependent extension of primers in PCR is catalyzed by a polymerizing agent in the presence of adequate amounts of four deoxyribonucleoside triphosphates (typically dATP, dGTP, dCTP, and dTTP) in a reaction medium comprised of the appropriate salts, metal cations, and pH buffering system. Suitable polymerizing agents are enzymes known to catalyze template-dependent DNA synthesis. For example, Thermus thermophilus (Tth) DNA polymerase, a thermostable DNA polymerase with reverse transcriptase activity is marketed by Roche Molecular Systems (Alameda, Calif.) PCR is most usually carried out as an automated process with a thermostable enzyme. In this process, the temperature of the reaction mixture is cycled through a denaturing region, a primer annealing region, and an extension reaction region automatically. Equipment specifically adapted for this purpose is commercially available from Roche Molecular Systems.

Most preferably, amplification of enteroviral RNA is carried out using a fluorescence based real-time detection method (e.g. SmartCycler®, Cepheid, or the ABI PRISM 7700 or 7900 Sequence Detection System [TaqMan®], Applied Biosystems, Foster City, Calif.) or similar system as described by Heid et al., (Genome Res 1996;6:986-994) and Gibson et al.(Genome Res 1996;6:995-1001). The output of the ABI 7700 (TaqMan® Instrument) is expressed in Ct's or “cycle thresholds”. A higher number of target molecules in a sample generates a signal with fewer PCR cycles (lower Ct) than a sample with a lower number of target molecules (higher Ct). By extension, given a set number of cycles, the level of fluorescence generated in the reaction will be indicative of the amount of amplification product which in turn, is a function of the amount template nucleic acid in the original biological sample. Therefore, real-time PCR also allows for the quantification of template DNA in the original biological sample. Preferably, the hydrolysis or TaqMan® probes for the oligonucleotide primer pair SEQ ID NO: 1 and 2, are SEQ ID NOs: 3-5.

One of skill will recognize, however, that the oligonucleotides of the invention are useful for detecting enteroviral RNA by any known method, such as ligase chain reaction (LCR) or self-sustained sequence replication, each of which provides sufficient amplification. Additionally, the present invention envisages the quantification of enteroviral RNA via use of a PCR-free systems employing, for example fluorescent labeled probes similar to those of the Invader® Assay (Third Wave Technologies, Inc.).

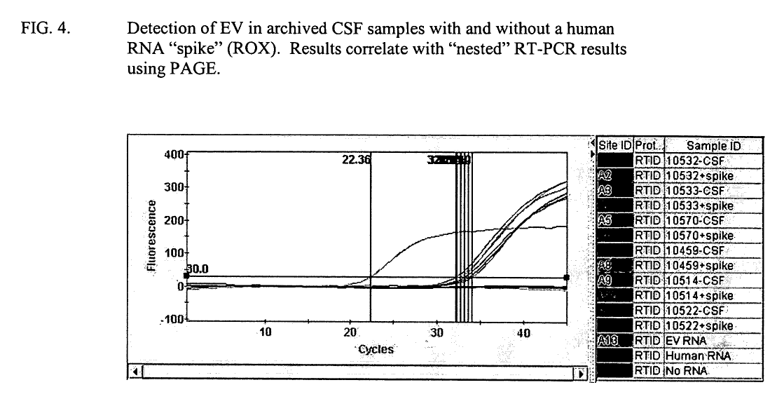

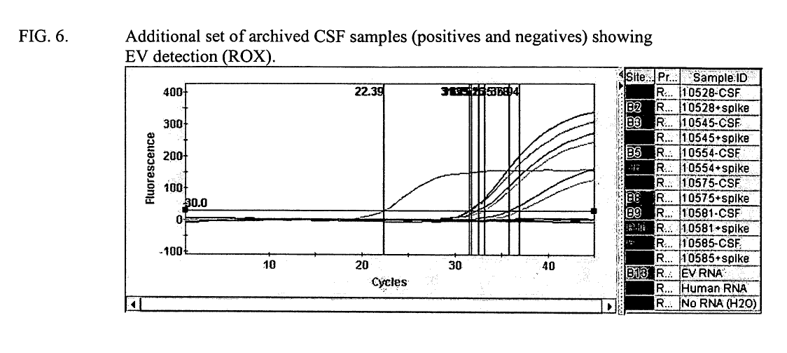

As used herein, an “internal control gene” is meant to include any constitutively or globally expressed gene whose mRNA transcripts enable an a control for variations in RNA recovery. In certain embodiments of the invention were the biological sample contains insufficient cellular material to as a source of internal control mRNA, the sample may be “spiked” with a predetermined amount of RNA to control for reverse transcription and amplification efficiency. “Internal controls” can include, but are not limited to premeasured RNA or mRNA transcripts of the cyclophilin gene, β-actin gene, the transferrin receptor gene, GAPDH gene, and the like. Most preferably, the internal control gene is β-actin gene as described by Eads et al., Cancer Research 1999; 59:2302-2306. See FIGS. 4-7.

Another aspect of the invention relates to a method of identifying compounds capable of inhibiting enteroviral growth. The method generally entails infecting a tissue culture with an enterovirus and then contacting a portion of the infected tissue culture with a compound suspected of being capable of inhibiting enteroviral growth. Next nucleic acids are isolated from the portion of the infected tissue culture contacted by the candidate compound. As a control, nucleic acids are also isolated from a portion of the remainder of the infected tissue culture not contacted by the candidate compound. Next, RT-PCR is performed on both nucleic acid samples in parallel. Preferably, SEQ ID NO: 1 or an oligonucleotide substantially identical thereto is used as the forward primer and SEQ ID NO: 2 or an oligonucleotide substantially identical thereto is used as the reverse primer. A decrease in an amplification product in the nucleic acid sample derived from the treated tissue culture relative to an amount amplification product in the nucleic acid sample derived from to the control indicates that a candidate compound is capable of inhibiting enteroviral growth. Prefereably, tissue culture comprises cells derived from from the group consisting of HEL, RMK, BGMK, MK, BGM, LLC-MK2, Vero, Hep-2, Rhadomyosarcoma, and new born mice. Additionlly, the term “tissue culture” as used herein is not limited to in vitro uses. The term also encompasses live animals that act as incubators for enteroviral growth such as suckling mice, rats or other mammals.

### EXAMPLE 1

Real-Time RT-PCR Procedure for Enterovirus

Preparation of template RNA/DNA: For EV RT-PCR, 200 ul of cerebral spinal fluid (CSF), serum, plasma, or pericardial fluid is processed using the Qiagen Viral RNA kit according to the manufacturers instructions. Combined RNA/DNA isolation is achieved for all biological samples using the Qiagen Viral RNA mini kit (catalog# 52906) according to manufacture's instructions. Total nucleic acids are recovered without a DNase treatment step, such that DNA as well as RNA targets can be analyzed. For tissues, 25-50 mg of tissue is disrupted in Buffer AVL contained in the Qiagen Viral RNA kit and processed as the sample types mentioned above. Nucleic acid without DNase treatment is eluted 2× using a single 60 ul volume of Buffer AVE. A 5.0 ul volume is used for RT-PCR.

Primer and probe design: Primers were designed from conserved regions of the 5′-untranslated region (5′-UTR) of the single-stranded RNA enterovirus genome. EV amplicons generated using SEQ ID NOs: 1 and 2, fall into 2 sequence classes due to serotype-specific polymorphisms. Therefore, SEQ ID NOs: 3 and 4 may be used to detect both classes of amplicons.

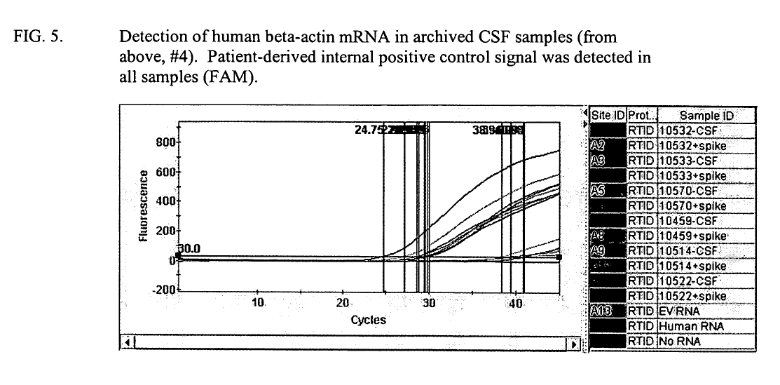

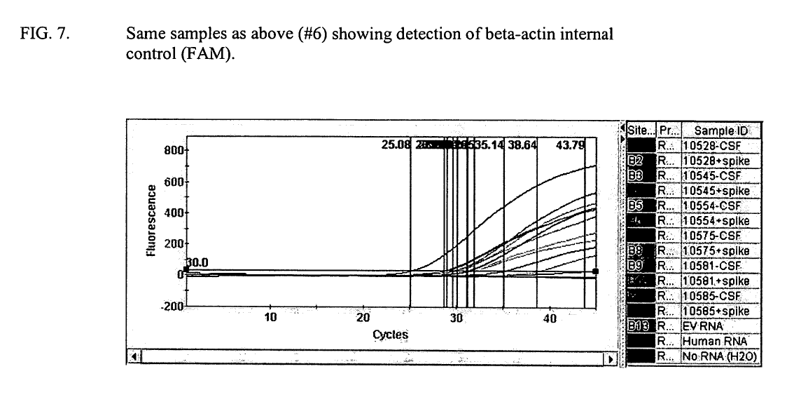

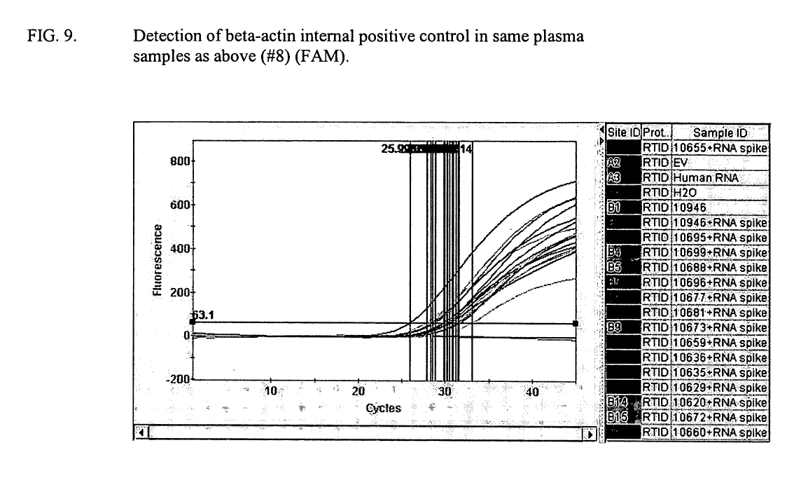

Internal control: The EV RT-PCR assay includes primers and FAM-labeled probe specific for human cytoplasmic beta-actin mRNA. This target is detectable in CSF (FIGS. 5 and 7), plasma (FIG. 9), and pericardial fluid due the presence of human cells. For these sample types, and especially serum, a second reaction is run in parallel containing a purified human RNA “spike” in a 1.0-ul volume. The internal positive control must be detectable in the “spiked” reaction. However, for the non-serum samples the internal control is routinely detected in the “unspiked” reaction and indicates successful extraction and RT-PCR performance.

Amplification: Exemplary reaction conditions are outlined in the table below:

TABLE 2
Master mix using individual reagents

| Stock Conc. | Volume | Final |
|---|---|---|
| Primers (5 μM) | 2.0 ul | 0.4 uM |
| Probe A (10 μM) | 1.0 ul | 0.4 uM |
| Probe B (10 uM) | 1.0 ul | 0.4 uM |
| H₂0 (PCR grade) | 2.5 ul | |
| SuperScript | 1.0 ul | |
| One-Step RT-PCR | | |
| Enzyme Mix | | |
| SuperScript 2X | 12.50 ul | 1X |
| Reaction buffer | | |
| Template | 5.0 ul | |

The manufacturer does not provide the units for the enzymes used in SuperScript ™One-Step RT-PCR with Platinum Taq. In any case, a determination of the amount of enzyme needed in an amplification reaction is well within the ordinary skill in the art. The 2× reaction buffer supplied with this enzyme mix yields a final concentration of 0.2 mM of each dNTP, and 1.2 mM MgSO₄. Additional MgSO₄ is not necessary for this assay.

TABLE 3
Exemplary Thermocycler Parameters

| Cycles | Stage | Temperature | Time | |
|---|---|---|---|---|
| 1 | RT step | 55° C. | 1800 | sec |
| 1 | Initial hold | 95° C. | 120 | sec |
| 45 | Denature | 95° C. | 15 | seconds |
| | Anneal | 55° C. | 15 | seconds |
| | Extend | 72° C. | 15 | seconds |
| 1 | Melt | 60° C. to 95° C. | 0.2° | C./sec |

The optics were turned on during the anneal step.

Parameters for Data Analysis: Parameters for Data Analysis: General description of procedures including: Dye Set-EV probe-ROX; internal control-FAM, Data Analysis Settings for the Smart Cycler® Instrument, Ct Analysis (primary), Manual 30.0 Background Subtract ON, Boxcar Averaging set at 0.

### EXAMPLE 2

Results

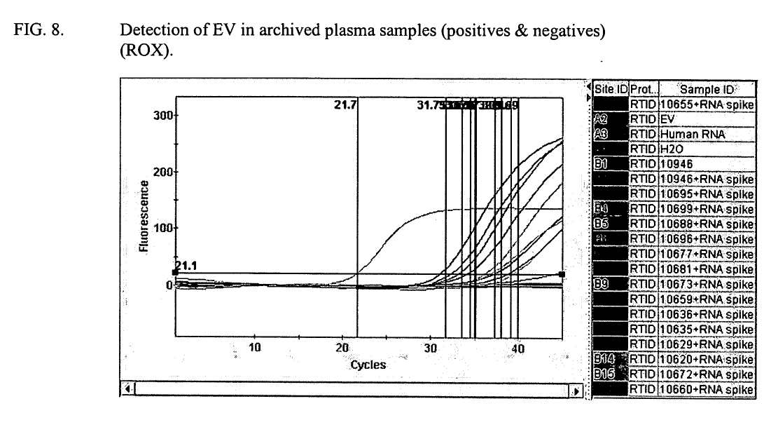

Sample Processing: The inclusion of primers and probe for the ubiquitous human beta-actin mRNA provides a patient-derived internal positive control for all reactions using plasma, swabs, and bodily fluids containing human cells. As mentioned above, serum testing includes a “spiked” reaction run in parallel. However, serum is not the preferable starting point, unless serum is the only sample type available from the lab. The protocol for preparing plasma from whole blood plasma ensures that residual lymphocytes will yield the beta-actin positive control signal (FIGS. 8-9). The Qiagen Viral RNA kit invariably provides intact, high quality RNA from all sample types tested.

Specific versus non-specific probes: The protocols disclosed herein preferably employ an amplicon specific probe for product detection. SyberGreen staining is not prefered due to the possibility of background signal. In addition, the method described herein are adaptable to multiplex PCR which necessitates the use of target-specific probes.

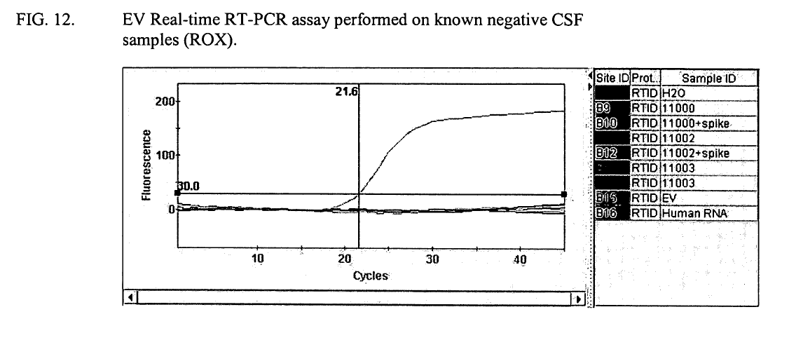

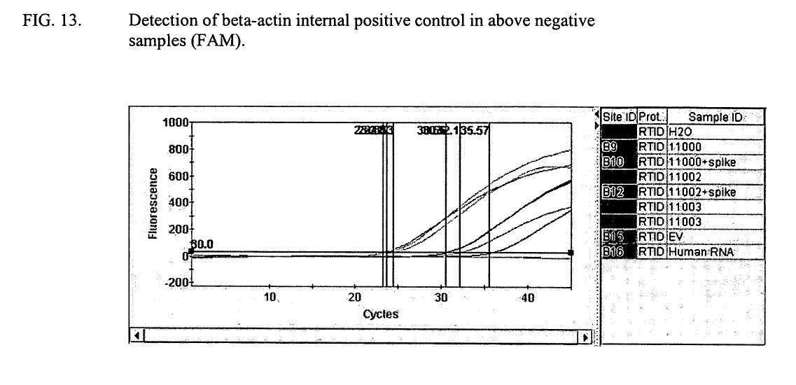

Quality Control: Patient assays were run in duplicate, with the option of the second reaction containing a human RNA “spike” as necessary. All assays include an EV external positive control containing an aliquot of EV Armored RNA (Ambion, Inc.) used according to the manufacturer's instructions. Two negative controls are included, 1) purified EV-negative human RNA extracted from control blood to test for beta-actin RT-PCR signal, and 2) a water blank to test for master mix quality. The patient derived beta-actin signal should be detectable in the absence of EV signal before an assay can be scored as “Not Detected” for EV (FIGS. 12-13). In the case of serum testing, or CSF samples that do not contain human cells i.e., CSF supernatant, the “spiked” sample should preferably yield the beta-actin signal in order to be scored as negative (See FIGS. 4-7).

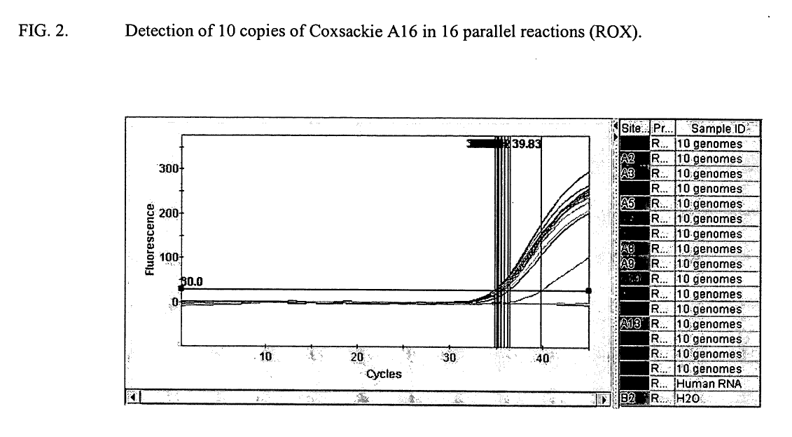

Sensitivity of the Assay: Preferably, the EV RT-PCR assay has a sensitivity adjusted to detect 10 EV RNA genomes equivalents in a background of about 2-4 ug of human RNA per reaction (FIG. 2). Using the primer and probe conditions stated above, 96 reactions containing 10 EV genomes in human RNA must be detectable in 96/96 assays run in parallel.

During validation, primer optimization is assessed independent of probe performance. Primers are optimized by performing reactions in the Smart Cycler in absence of probe and products are analyzed by high resolution PAGE. The lowest primer concentration yielding desired sensitivity in the absence of background is then tested in the presence of probe and fluorescence is monitored. Probe concentration is then adjusted such that the desired sensitivity is achieved with the lowest probe input.

Assay Notes

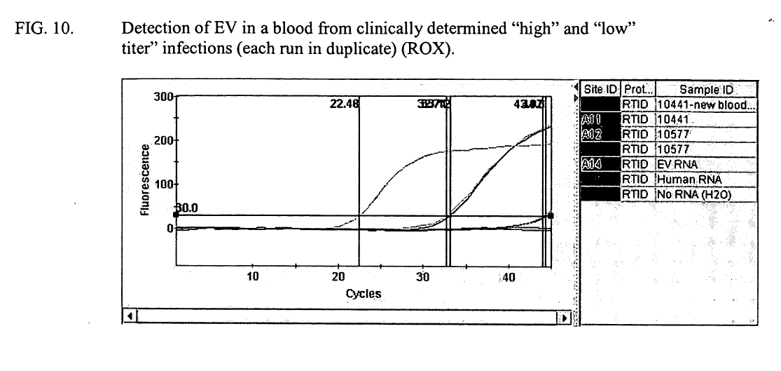

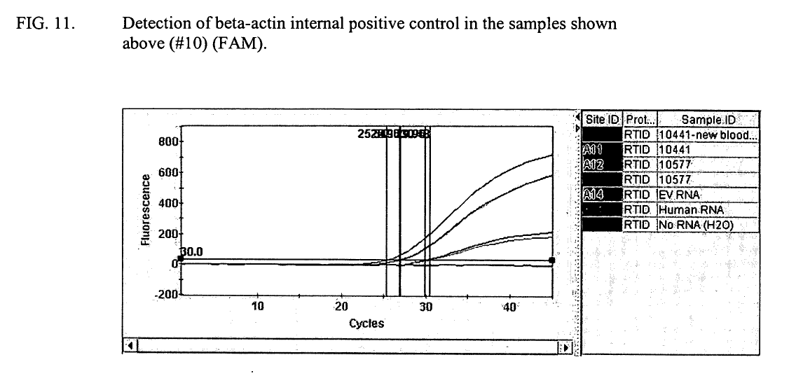

Quantity of archived samples: 64 known-positive and 64 known-negative archived samples were used, covering ½ plasma and ½ CSF samples. (FIGS. 4-13)

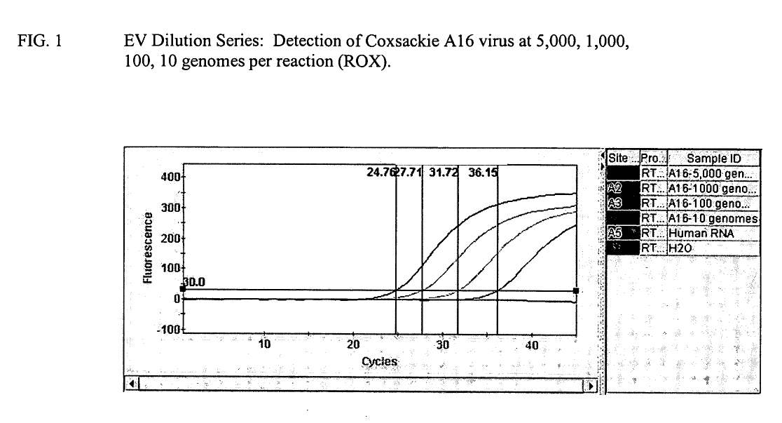

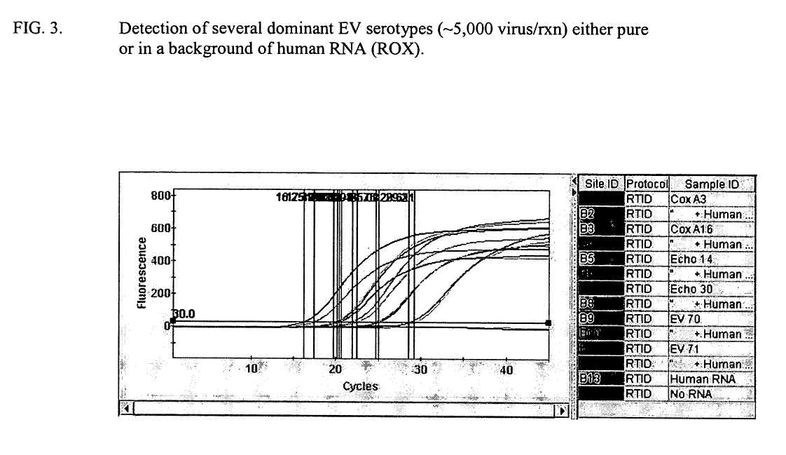

Types of samples used to optimize and validate assay: Enterovirus cultures of known titer were obtained from ATCC (Manassas, Va.). Coxsackie A16 was used as our reference strain for test validation (FIGS. 1 and 2). The other strains tested during validation included: Coxsackie A3, A16, echovirus 14, echovirus 30, enterovirus 70, enterovirus 71 (FIG. 3), Coxsackie A21, Coxsackie A9, Coxsackie B1, echovirus 6, echovirus 3, echovirus 9, echovirus 7, echovirus 11. All serotypes listed were detected at a 1:10,000 or 1:100,000 dilution.

In this disclosure there are described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

1. An isolated oligonucleotide of the sequence SEQ ID NO: 1.

2. An isolated oligonucleotide that hybridizes the complement of SEQ ID NO: 1 under stringent conditions and is capable of amplifying reverse transcribed enteroviral RNA when used in conjunction with SEQ ID NO: 2 in a polymerase chain reaction.

3. An isolated oligonucleotide of the sequence of SEQ ID NO: 1, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 2 in an polymerase chain reaction.

4. An isolated oligonucleotide of the sequence SEQ ID NO: 2.

5. An isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction.

6. An isolated oligonucleotide of the sequence of SEQ ID NO: 2, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 1 in an polymerase chain reaction.

7. An isolated oligonucleotide having the sequence of SEQ ID NO: 3 or a sequence wherein wherein about one to about three nucleotides are added or removed from the ′5 end and/or about one to about three nucleotides are added or removed from the 3′ end of SEQ ID NO: 3.

8. A kit for detecting enteroviral RNA comprising a first isolated oligonucleotide of SEQ ID NO: 1 and a second oligonucleotide of any one of claims 4 to 6.

9. A kit for detecting enteroviral RNA comprising a first isolated oligonucleotide of SEQ ID NO: 2 and a second oligonucleotide of any one of claims 1 to 3.

10. A kit for detecting enteroviral RNA comprising a first isolated oligonucleotide of SEQ ID NO: 1 and a second oligonucleotide of SEQ ID NO: 2.

11. A kit for detecting enteroviral RNA comprising a first oligonucleotide selected from the group consisting of:
(A) an isolated oligonucleotide of the sequence SEQ ID NO: 1;
(B) an isolated oligonucleotide that hybridizes the complement of SEQ ID NO: 1 under stringent conditions and is capable of amplifying reverse transcribed enteroviral RNA when used in conjunction with SEQ ID NO: 2 in an polymerase chain reaction; and
(C) an isolated oligonucleotide of the sequence of SEQ ID NO: 1, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 2 in an polymerase chain reaction;
and a second oligonucleotide selected from the group consisting of
(A) an isolated oligonucleotide of the sequence SEQ ID NO: 2;
(B) an isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction; and
(C) an isolated oligonucleotide of the sequence of SEQ ID NO: 2, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 1 in an polymerase chain reaction.

12. The kit of claims 10 or 11 further comprising at least one probe oligonucleotide selected from the group consisting of:
(A) SEQ ID No: 3 or a sequence wherein about one to about three nucleotides are added or removed from the ′5 end and/or about one to about three nucleotides are added or removed from the 3′ end of the sequence set forth in SEQ ID NO: 3;
(B) SEQ ID No: 4 or a sequence wherein about one to about three nucleotides are added or removed from the ′5 end and/or about one to about three nucleotides are added or removed from the 3′ end of the sequence set forth in SEQ ID NO: 4;
(C) SEQ ID No: 5 or a sequence wherein about one to about three nucleotides are added or removed from the ′5 end and/or about one to about three nucleotides are added or removed from the 3′ end of the sequence set forth in SEQ ID NO: 5.

13. The kit of claims 10 or 11 further comprising a PCR reaction buffer and DNA polymerase enzyme.

14. A method of detecting the presence of enteroviral RNA in a biological sample comprising:
(A) obtaining a biological sample from an organism;
(B) isolating nucleic acids from said sample;
(C) performing a polymerase chain reaction on said isolated nucleic acids using a first isolated oligonucleotide selected from the group consisting of:
(i) an isolated oligonucleotide of the sequence SEQ ID NO: 1;
(ii) an isolated oligonucleotide that hybridizes the complement of SEQ ID NO: 1 under stringent conditions and is capable of amplifying reverse transcribed enteroviral RNA when used in conjunction with SEQ ID NO: 2 in an polymerase chain reaction; and
(iii) an isolated oligonucleotide of the sequence of SEQ ID NO: 1, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 2 in an polymerase chain reaction;
and a second oligonucleotide selected from the group consisting of
(i) an isolated oligonucleotide of the sequence SEQ ID NO: 2;
(ii) an isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction; and
(iii) an isolated oligonucleotide of the sequence of SEQ ID NO: 2, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 1 in an polymerase chain reaction,
(D) correlating a presence of an amplification product from said polymerase chain reaction with the presence of enteroviral RNA in said sample.

15. The method of claim 14 wherein, the biological sample is selected from the group consisting of a tissue sample, whole blood or serum, sputum, stool, urine, semen, pericardial fluid, nasopharyngeal/throat swabs, cerebrospinal fluid (CSF), and amniotic fluid.

16. The method 14 wherein the organism is a patient suspected of having being infected by an enterovirus.

17. The method of claim 14 where the polymerase chain reaction is a real-time polymerase chain reaction.

18. The method of claim 14 wherein a portion of an internal control DNA is amplified at the same time as said enteroviral RNA.

19. The method of identifying compounds capable of inhibiting enteroviral growth comprising:
(A) infecting a tissue culture with an enterovirus to obtain an infected tissue culture;
(B) contacting a portion of said infected tissue culture with a compound suspected of being capable of inhibiting enteroviral growth;
(C) isolating nucleic acids from the portion of said infected tissue culture contacted by said compound to obtain a first nucleic acid sample and from a portion of the remainder of the infected tissue culture not contacted by said compound to obtain a second nucleic acid sample;
(D) performing polymerase chain reaction on said first and said second nucleic acid samples, using a first isolated oligonucleotide selected from the group consisting of:
(i) an isolated oligonucleotide of the sequence SEQ ID NO: 1;
(ii) an isolated oligonucleotide that hybridizes the complement of SEQ ID NO: 1 under stringent conditions and is capable of amplifying reverse transcribed enteroviral RNA when used in conjunction with SEQ ID NO: 2 in an polymerase chain reaction; and
(iii) an isolated oligonucleotide of the sequence of SEQ ID NO: 1, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 2 in an polymerase chain reaction;
and a second oligonucleotide selected from the group consisting of
(i) an isolated oligonucleotide of the sequence SEQ ID NO: 2;
(ii) an isolated oligonucleotide that hybridizes the complement of SEQ ID NO: 2 under stringent conditions and is capable of amplifying reverse transcribed enteroviral RNA when used in conjunction with SEQ ID NO: 1 in an polymerase chain reaction; and
(iii) an isolated oligonucleotide of the sequence of SEQ ID NO: 2, wherein from about one to about three nucleotides are added or removed from the ′5 end and/or from about one to about three nucleotides are added or removed from the 3′ end, respectively, and wherein the oligonucleotide is capable of amplifying reverse transcribed enteroviral RNA when used in conjunction with SEQ ID NO: 1 in an polymerase chain reaction,
(D) whereby a decrease in an amplification product in the first nucleic acid sample relative to the second nucleic acid sample indicates that the compound is capable of inhibiting enteroviral growth.

20. The method of claim 19 wherein said tissue culture comprises cells derived from from the group consisting of HEL, RMK, BGMK, MK, BGM, LLC-MK2, Vero, Hep-2, Rhadomyosarcoma, and new born mice.

21. An isolated oligonucleotide having the sequence of SEQ ID NO: 4 or a sequence wherein wherein about one to about three nucleotides are added or removed from the ′5 end and/or about one to about three nucleotides are added or removed from the 3′ end of SEQ ID NO: 4.

22. An isolated oligonucleotide having the sequence of SEQ ID NO: 5 or a sequence wherein wherein about one to about three nucleotides are added or removed from the ′5 end and/or about one to about three nucleotides are added or removed from the 3′ end of SEQ ID NO: 5.
